(19) United States
(12) Patent Application Publication
(10) Pub. No.: US 2018/0166162 A1
(43) Pub. Date: Jun. 14, 2018

(54) MEDICAL SYSTEM

(71) Applicant: TOSHIBA MEDICAL SYSTEMS CORPORATION, Otawara-shi (JP)

(72) Inventors: Hikaru FUTAMI, Nasushiobara-shi (JP); Kenichi NIWA, Otawara-shi (JP); Kohki FUJII, Nashushiobara (JP)

(73) Assignee: TOSHIBA MEDICAL SYSTEMS CORPORATION, Otawara-shi (JP)

(21) Appl. No.: 15/878,998

(22) Filed: Jan. 24, 2018

Related U.S. Application Data
(62) Division of application No. 13/824,039, filed on Mar. 15, 2013, now abandoned.

(30) Foreign Application Priority Data
Dec. 15, 2010 (JP) 2010-278791

(51) Int. Cl. G16H 15/00; G06Q 50/22; G16H 10/60; G06Q 10/10

(52) U.S. Cl. CPC G16H 15/00; G06Q 10/10; G16H 10/60; G06Q 50/22

(57) ABSTRACT

The medical system of the embodiment comprises a recording unit, management unit, extracting unit, searching unit, and display unit. The recording unit records medical information by associating it to the classification thereof. The management unit associates and manages a plurality of situation sections in order to differentiate medical examination situations when medical care is received and a plurality of classifications of medical information. The extracting unit extracts the classification of medical information corresponding to particular situations among a plurality of situation sections based on the plurality of situation sections associated and a plurality of classifications of medical information by the management unit. The searching unit searches the classification of medical information not recorded in the recording unit among the classifications of medical information extracted by the extracting unit. The display unit displays the classification of medical information searched by the searching unit.

### TECHNICAL FIELD

Embodiments of the present invention is related to a medical system that uses the medical information of patients obtained when medical care is received.

### BACKGROUND

The medical system involves a system of dividing the processing of information including the medical information of patients and a system of concentrating all processes in one computer. A client server system is used as an example of the former, while a stand-alone system is used as an example of the latter.

Here, when mentioning “medical system” or simply “system,” they are composed so as to include the computer mentioned above and other medical equipment. The computer and medical equipment are connected by a network. Examples of the medical equipment include a medical image storage device that records images of patients, etc., taken using medical imaging devices such as an X-ray computed tomography (CT) scanner and/or magnetic resonance imaging (MRI) scanner, etc., a data base that records supplementary information of patients and the location of images, etc., a medical image reference device that refers the recorded image to users, and, for example, an interpretation report preparing device that prepares survey reports on medical findings regarding the image. Moreover, there are times in which a survey report prepared based on the images taken with the X-ray CT scanner is specifically referred to as an interpretation report.

The medical information of patients is taken into such a system and utilized. Here, when mentioning “taken into the system,” this may refer to recording in the medical image storage device and the database provided in the system. Moreover, when mentioning “utilize,” it may refer to one or a combination of two or more among preparing the survey report based on the medical information, recording the medical information in the medical image storage device, recording the medical information and the location thereof in the database, and referring to the medical information using the medical image reference device.

Generally, the medical information of patients to be taken into the system is associated with the medical examination situations when patients receive medical care. Therefore, for example, based on the association thereof, the user (operator) takes the medical information into the system.

An example of the medical information of patients is information that is prepared in the medical system when the patient receives medical care and automatically taken into the system. For example, medical images of patients taken by the CT scanner are automatically recorded in the medical image storage device, and the supplementary information of patients and location of the medical images are automatically recorded in the database.

The medical information is automatically taken into the system. Therefore, taking into the system is possible without the user (operator) referring to the association of the medical information and medical examination situations, and subsequently, may be used in the system.

Furthermore, the record form of the medical information of patients comprises information such as medical records (clinical charts), test images, and survey reports (interpretation reports) in addition to general-purpose files, for example, word files by Microsoft, PDF (portable document format) files, etc.

However, mainly due to the condition of the recording medium of the medical information, there are times in which the medical information cannot be automatically taken in. For these instances, the user (operator) refers to the association of the medical information and medical examination situation, and takes the information into the system.

Examples of information that cannot be automatically taken into the system include information that is not made into electronic data such as on paper media, and information brought in from outside the system by media such as X-ray film, CD-R, etc. Furthermore, there is information that is not set to be automatically taken into the system, such as electrocardiogram (ECG) data.

The information from paper media is computerized by scanner uptake from OCR (Optical Character Recognition) and the information from X-ray films is computerized using a digitizer, both of which are taken into the medical system. Furthermore, storage media such as CD-R, etc., are taken into the system by a reader. Furthermore, the electrocardiogram (ECG) data is taken into the system from input operations by the operator (user).

A further concrete example is described below. Upon consultation as an outpatient and/or admission to a hospital of the patient through referral from hospital A to hospital B, a letter of introduction via a paper medium provided to the patient, test images using films (there are cases in which these are brought in with CD-R), and survey reports by paper media (interpretation reports) (there are cases in which these are brought in with CD-R) are handed from hospital A to hospital B. At this time, in hospital B, information other than that of hospital B may be taken into the medical system operated in hospital B in order to manage in an integrated manner the information brought in.

Moreover, written consent may be obtained in paper prior to surgery and/or treatment of relevant patient and particular tests; the written consent is also computerized by scanner uptake and taken into the medical system at times. In this manner, the information from paper, film, etc., is computerized and taken into the medical system; thereby, simplifying usage in the system.

As mentioned above, medical information that cannot be automatically taken in is, for example, taken into the system by the user (operator) converting and computerizing this or conducting input operations with reference to the association of medical information and taking it into the system.

As a method of taking into the system information that is not computerized such as with paper media, etc., a barcode adding a predetermined ID code to the subject to be taken into the system is output, and by adding the barcode to the information to be taken in, information may be taken into the system while preventing incorrect input (patent literature 1).

As a method of extracting the information accumulated into the system based on the medical examination situation, the system may be searched and by displaying the list thereof based on the recorded setting, the information of the patient may be collected (patent literature 2).

### PATENT DOCUMENT

[Patent Document 1] Japanese published unexamined application 2007-4342

[Patent Document 2] Japanese Unexamined Patent Application Publication 2009-193157

### SUMMARY OF THE INVENTION

### Problems to be Solved by the Invention

However, health care providers (medical care physicians, radiologists) often refer to images, reports, written consents, etc., upon medical care, diagnosis, and tests. For example, written consents, etc., is information that becomes the basis of medical practices when conducting tests. That is to say, the information to be taken into the system is present due to medical examination situations.

There is no problem if images and reports are automatically incorporated as electronic data. However, when these are brought in from outside the system via the media of film, CD-R, etc., or when it is a paper medium that is not taken into the system, problems are caused such as, for example, surgery, treatment, tests, etc., cannot be conducted without written consent, comparative reading cannot be conducted between the test conducted in the relevant institution and the image brought in through referral, etc. (medical practices are delayed.).

Medical practices may be continued if the film, CD-R, and paper media are referred to. However, only several originals are present, so one must go to the location at which the references thereof are stored. Moreover, using references is difficult when they are in use by other doctors. Moreover, use by other doctors will become possible if a copy thereof is made. However, this contradicts the purpose of managing information taken into the hospital in an integrated manner.

In patent literature 1, though it is a method used to prevent mis-registration, the information of the relevant patient to be taken in must be specified by the operator themselves searching the system. Moreover, a method is used of acquiring uniqueness of the information to be taken in and the information taken in, by taking in the information with the barcode added. However, there is no determination of the conformity (validity) of the information to be taken in and the information that was taken in. There is a danger of the information to be taken in and the information that was taken in being of an incorrect combination due to a mistake upon adding the barcode.

In patent literature 2, there was a problem in that although necessary information may be searched based on the medical examination situation of the patient and displayed, information that does not clearly exist cannot be comprehended; moreover, it is realized that the information that should be taken in is not present at a certain stage of medical care, and the operator has to take in the information.

This embodiment solves the problems mentioned above, with the purpose of determining the necessary medical information according to the medical examination situation of the patient, searching the system, and if the medical information is not taken into the system, informing the operator of medical information that should be taken in.

### Means of Solving the Problem

To solve the problem mentioned above, the medical system of the embodiment comprises a recording unit, management unit, extracting unit, searching unit, and display unit. The recording unit records medical information by associating them to the classification thereof. The management unit associates and manages a plurality of situation sections in order to differentiate medical examination situations when medical care is received along with a plurality of classifications of medical information. The extracting unit extracts the classification of medical information corresponding to particular situations among the plurality of situation sections based on the plurality of situation sections and a plurality of classifications of medical information associated by the management unit. The searching unit searches the classification of medical information not recorded in the recording unit among the classifications of medical information extracted by the extracting unit. The display unit displays the classification of medical information searched by the searching unit.

### BRIEF DESCRIPTION OF THE DRAWINGS

FIG. 1 is a block diagram of the medical system related to the first embodiment.

FIG. 2 is a diagram showing an example of the combination of medical information and the situation section recorded in the database.

FIG. 3 is a diagram showing an example of the medical information recorded in the storage unit.

FIG. 4 is a diagram showing an example of the combination of the classification of medical information and the template.

FIG. 5 is a diagram showing a presented example of the categorization of the extracted medical information.

FIG. 6 is a flow chart showing the actions from extraction to list display.

FIG. 7 is a diagram showing an example of the template displayed by specifying the template example in the medical system related to the second embodiment.

FIG. 8 is a flow chart showing the actions from medical information input to recording of the medical information.

FIG. 9 is a diagram showing an example of the combination of input pattern and the classification of medical information recorded in the database in the medical system related to the third embodiment.

FIG. 10 is a diagram showing the input example of a survey report (interpretation report).

FIG. 11 is a flow chart showing the actions of text string extraction/decision.

FIG. 12 is a diagram conceptually showing an example of the association of text strings and medical information in the medical system related to the fourth embodiment.

FIG. 13 is a diagram showing other examples of the association of the text strings and medical information.

FIG. 14 is a diagram showing an example of when the data subject for association is not taken in.

FIG. 15 is a flow chart showing the actions of link generation.

FIG. 16 is a diagram showing an example of the database of the medical examination record in the medical system related to the fifth embodiment.

FIG. 17 is a diagram showing an example of the master database.

FIG. 18 is a diagram showing an example of the database of the medical examination.

FIG. 19 is a diagram showing an example of an item of medical information displayed on the monitor.

FIG. 20 is a diagram showing an example of the database of fixed phrases.

### MODES FOR CARRYING OUT THE INVENTION

Next, various embodiments of the medical system are described with reference to each diagram. First, this medical system clearly shows to the user (operator) what information is taken into the system, allowing for the full equipping of information to the system (solidity of information). Secondly, the validity of the information to be taken in is differentiated by preventing different information from being taken into the system by mistake (validity of information). Thirdly, the information taken into the system is sufficiently put to practical use (usability of information).

The basic configuration of this medical system comprises a recording unit that associates the medical care condition when medical care is received with the situation section differentiated by the conditions thereof and that records the medical information obtained under the situation section. Here, when mentioning “recording unit,” this may refer to at least one among the medical image storage device that memorizes images, etc., of the patient, the database that records the supplementary information of the patient and the location of images, etc. Moreover, in the following description, to record medical images in the recording unit refers to taking in medical images to the system.

Each of the following embodiments describes the medical system attempting each of: the solidity of the information mentioned above, the validity of information, and the usability of information.

### First Embodiment

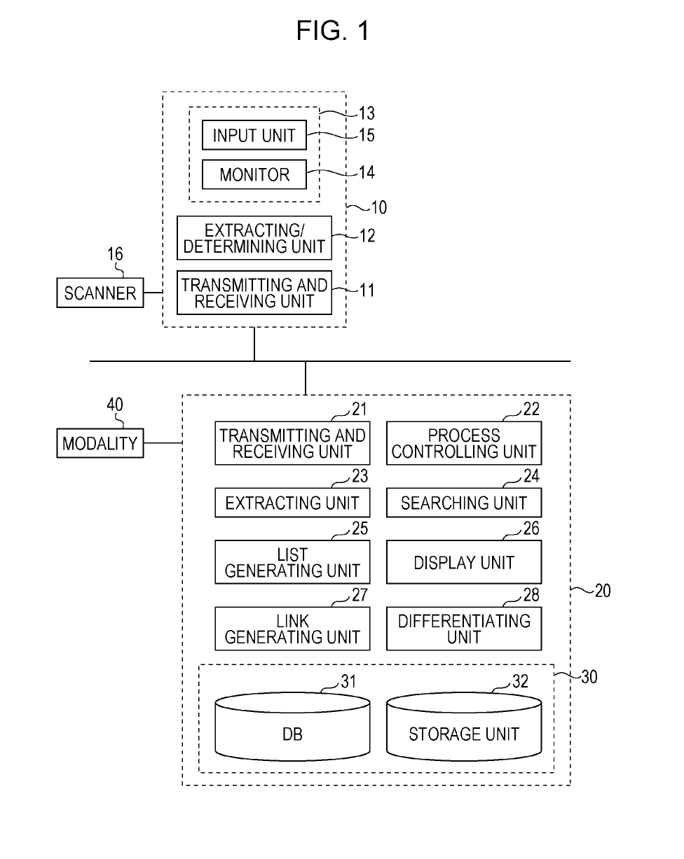

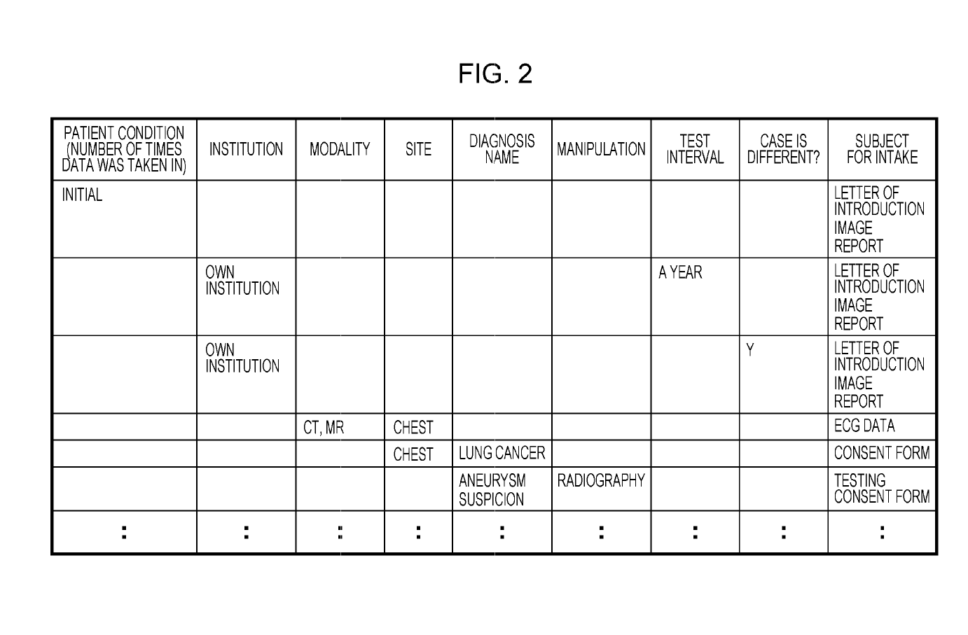

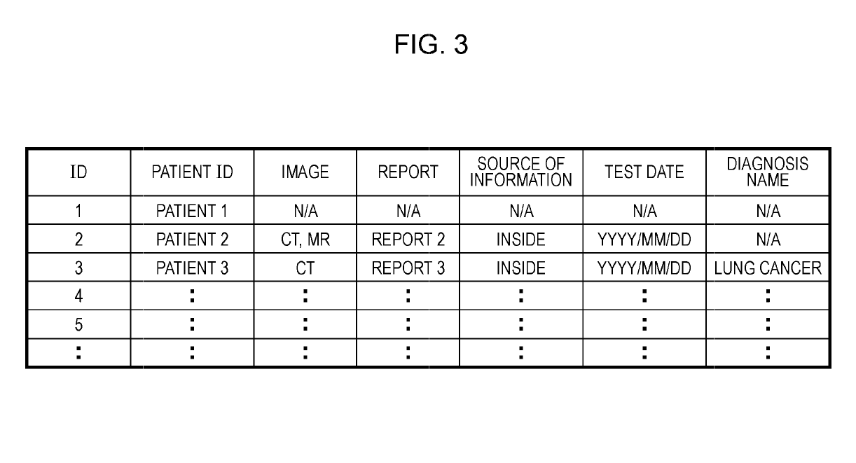

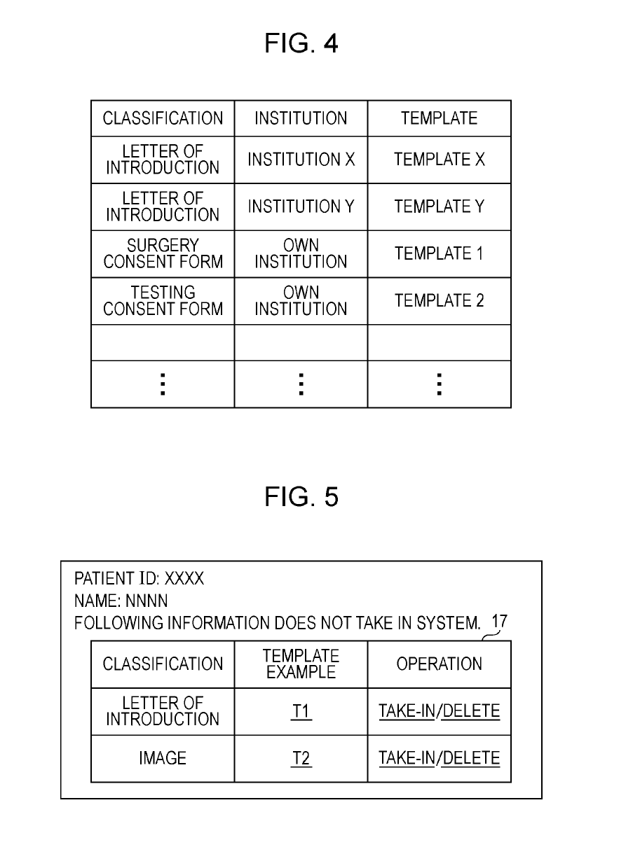

First, the medical system attempting solidity of information is briefly explained with reference to FIG. 1. FIG. 1 is a block diagram of the medical system, FIG. 2 is a diagram showing an example of the combination of the associated medical information and situation section, FIG. 3 is a diagram showing an example of the medical information recorded in the storage unit, FIG. 4 is a diagram showing a presentation example of the classification of extracted medical information, and FIG. 5 is a diagram showing a presentation example of the classification of extracted medical information.

This medical system comprises a database (Database) 31 in which the association between the situation section and the classification of medical information is established in advance, a searching unit 24 that searches whether or not the situation section and the associated classification of medical information are taken into the system, and a monitor 14 that informs of the results searched using the searching unit 24. Here, “situation section” refers to the differentiation of medical examination situations under the condition when the patient receives medical care. Furthermore, details on the condition are mentioned later. With the description below, there are times in which the “situation section” is referred to as a condition. Moreover, the classification of medical information is the type among test images, survey reports, letters of introduction, and other medical images; these are included in the medical information as, for example, supplementary information. Accordingly, the classification thereof may be differentiated based on the medical information.

A database 31 is an example of “management unit.” Moreover, the searching unit 24 is an example of “searching unit.” Furthermore, the monitor 14 is an example of “informing unit.”

By means of providing the database 31, searching unit 24, and monitor 14, it becomes possible to determine the necessary medical information according to the medical examination situation of the patient, search the medical system, and inform the user (operator) whether or not medical information is taken into the system.

Next, the database 31, which is an example of the management unit, is explained with reference to FIG. 2. As mentioned above, association of the situation section and the medical information is established in advance regarding the database 31. As an example of the condition to differentiate the medical examination situations when the patient receives medical examination to situation sections, whether there is difference in the number of times the medical information of the patient was taken in at one's own institution (number of times data was taken in), the institution at which the medical information was prepared, modality of obtaining medical information, the photographed site, diagnosis name, manipulation, test interval, and medical cases, are used.

The association of the situation section and classification of medical information is further explained with reference to FIG. 2. As the first situation section, there is “initial” regarding the number of times data is taken in. As the second situation section, there is the institution which is one's “own institution” and the test interval which is “a year.” Furthermore, as the third situation section, there is the institution which is one's “own institution” and is “Y” regarding whether the case is different. As the fourth situation section, there is the modality which is “CT and MR” and the site which is “chest.”

In each situation section of the first to the third, the classification of medical information (“subject for intake” in FIG. 2) of (“letter of introduction,” “images,” “report”) is respectively associated. Moreover, the classification of medical information of “ECG data” is associated with the fourth situation section. Moreover, association of the conditioned situation section and the classification of medical information may be newly determined and recorded in the database 31.

Next, the configuration of the medical system of this embodiment is briefly explained. Regarding the configuration of the medical system, the configuration of respective processing units and physical locations of respective processing units are not limited. Moreover, by integrating to one PC as software, a stand-alone type may be configured. In this embodiment, the medical system is entered as a server-client type system.

### (Server)

First, the configuration of a server 20 is briefly explained.

A transmitting and receiving unit 21 receives images from an imaging study device, transmits various data to a client 10, and receives data, etc. from the client 10. A process controlling unit 22 controls the processes of each processing unit. An extracting unit 23 extracts information pertaining to the medical care of the patient from the database 31. A searching unit 24 receives the outcomes from the extracting unit 23, searches the recording unit 30, and differentiates the presence of the extracted information.

A list generating unit 25 receives the search results from the searching unit 24 and generates a list of medical information classifications planned to be taken into the recording unit 30, with a warning text string informing with regard to the medical information that should be taken in. The generated list of medical information classifications and the warning text string are displayed on the monitor 14 of the client 10.

The database 31 manages the storage locations of images, interpretation reports, and files in addition to supplementary information (patient ID, test date, series ID, image ID, report ID, etc.). Furthermore, an interpretation report was raised as an example of a survey report, but it goes without saying that this embodiment is not limited to this, and may be widely applied to other survey reports. The same goes for the other embodiments. The storage unit 32 records the substantial images, reports, and files. Furthermore, when “recording unit 30” is mentioned in the description below, this may refer to at least one among the database 31 and the storage unit 32.

Next, the medical information of the patient recorded in the storage unit 32 is explained with reference to FIG. 3. The medical information of the patient is configured from respective information regarding a patient's ID, images, reports, information source, test date, and diagnosis name per ID.

For example, the medical information of ID “1” includes: “patient 1,” “N/A,” “N/A,” “N/A,” “N/A,” and “N/A.” Here, “N/A” refers to an undetermined value (No Assign). The medical information of ID “2” includes: “patient 2,” “CT,” MR,” “report 2,” “inside,” “YYYY/MM/DD,” and “N/A.” The medical information of ID “3” includes: “patient 3,” “CT,” “report 3,” “inside,” “YYYY/MM/DD,” and “lung cancer.”

Moreover, in this institution, when a patient receives medical care on a different test date, a different ID is attached even though it involves the same patient, respective information ranging from patient ID to diagnosis name is prepared for the ID thereof, and this information is recorded in the storage unit 32.

As in the above, due to configuring the database 31 and the storage unit 32, when the same patient undergoes the same medical care a plurality of times at the same institution on different test dates, in the institution thereof, this information is managed separately as the medical information of a patient with a different patient ID.

For example, when a patient that received medical care a year after receiving initial medical care in the same institution is hypothesized, with respect to the initial situation section, the classification of medical information that should be recorded in the recording unit 30 (to be recorded into the system) includes: “letter of introduction,” “images,” and “reports.” Moreover, with respect to the situation section following a year, the classification of medical information that should be recorded in the recording unit 30 also includes: “letter of introduction,” “images,” and “reports.” The searching unit 24 searches whether or not a “letter of introduction,” etc., is recorded in the recording unit 30 with respect to the initial situation section, and moreover, also searches whether or not a “letter of introduction,” etc., is recorded in the recording unit 30 regarding the situation section after a year.

Next, the model form recorded in the storage unit 32 is explained with reference to FIG. 4. Here, the “model form” refers to an example showing a predetermined manner of writing. Moreover, in the following explanation, the model form may be referred to as a “template.”

When the classification of medical information is a “letter of introduction,” “template X” of “institution X” and “template Y” of “institution Y” are recorded as the template of the institution. Moreover, when the classification of medical information is a “surgery consent form,” “template 1” of one's “own institution” is recorded as the template of the institution, and when the classification of medical information is a “testing consent form,” “template 2” of one's “own institution” is recorded as the template of the institution.

Upon taking medical information into the system, if, for example, when the classification of medical information is specified by the operation of the input unit 15, the template of the medical information of the specified classification is read from the storage unit 32 and displayed on the monitor 14. The user (operator) may compare the template of the displayed medical information and the classification (type) of medical information planned to be taken into the system, thereby determining the validity of the classification (type) of medical information planned to be taken in.

In the above, an example of the user determining the validity of the classification (type) of medical information when taking in medical information into the system was shown; however, the determining unit 28 may differentiate the validity of the classification (type) of medical information on behalf of the user. The determining unit 28 differentiates whether or not the classification (type) of medical information planned to be taken in to the recording unit 30 conforms to the template of the medical information that should be taken in. The template that becomes the source for differentiation is obtained from the storage unit 32. The determining unit 28 is an example of an “information determining unit.” Moreover, details regarding the differentiation of the validity of the classification (type) of medical information by the determining unit 28 are mentioned later.

### (Client)

Next, configuration of the client is briefly explained.

A transmitting and receiving unit 11 transmits and receives data from the server 20, transmits data to the server 20, obtains read data from the scanner 16 separately connected to the client 10, transmits and receives information from the extracting/determining unit 12, transmits information from the interface 13, or transmits the displayed information to the interface 13. The interface 13 comprises the monitor 14 and devices such as a keyboard, mouse, etc. The text string is displayed on the monitor 14 from input operations by the user to input units 15 such as the keyboard, mouse, etc. Moreover, due to the input operation by the user, information is displayed on the monitor, allowing for further specification.

The interface 13 receives requests from the transmitting and receiving unit 11 and displays the information generated by the list generating unit 25 on the monitor 14, allowing for specification. The interface 13 is an example of the “display unit.”

The extracting/determining unit 12 extracts the text string input by the input operation from the user together with determining whether or not the text string thereof corresponds with the classification of medical information.

Moreover, though not illustrated in FIG. 1, the server or the client comprises a function to prepare/store/refer to the interpretation reports.

Moreover, information communication between the client and the server is the TCP/IP (Transmission Control Protocol/Internet Protocol) transmission of the local standard, wherein, data is exchanged with a packet (standard unit when transmitting information). The information communication between the client and the scanner 16 is exchanged by a USB (Universal Serial Bus). Moreover, this communication is not limited to those mentioned above, and other existing specifications may also be applied.

### (Action)

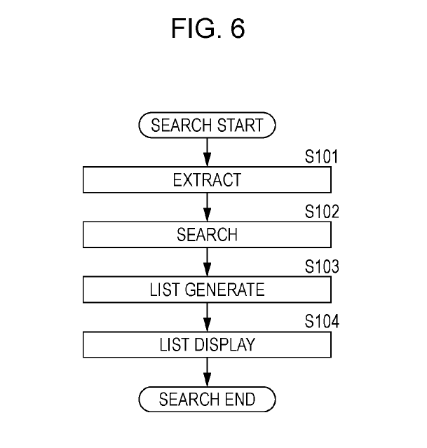

Next, as an example of the medical system actions, the case of a patient 1 that underwent a medical examination through the referral of another institution is explained with reference to FIGS. 2 to 6. FIG. 5 is a diagram showing a presented example of the categorization of the extracted medical information, while FIG. 6 is a flow chart showing the actions from extraction to list display. Here, “patient 1” is the identification number attached as a patient ID shown in FIG. 3.

### (S101)

The extracting unit 23 extracts from the database 31 the information that should be taken into the system from the relationship shown in FIG. 2 and the condition of the relevant patient. In the case of patient 1, images are transmitted to the system from the modality 40, this is received by the transmitting and receiving unit 21, and the supplementary information of images is saved in the database 31 while the entity is saved in the storage unit 32. At this time, it is shown that the source of information of images is inside. Moreover, the system receives the information of patient 1 for the first time, so this becomes the first time in which the data is handled. From the relationship of FIG. 2, the extracting unit 23 extracts the letter of introduction, images, and reports as information that should be taken into the system.

### (S102)

Subsequently, the processes shift to the searching unit 24. In the searching unit 24, whether or not the information extracted at the extracting unit 23 is recorded in the recording unit 30 is searched. In the case of patient 1, the classification of medical information that should be recoded in the recording unit 30 under the situation section differentiated by conditions, includes: a letter of introduction, images, and reports (refer to FIG. 2). When the letter of introduction and images taken in from outside (not occurring in the medical institution) are not recorded in the recording unit 30, the search results thereof are recorded in the database 31.

### (S103)

The list generating unit 25 makes a list of the classifications of medical information (letter of introduction and images) that are not recorded in the recording unit 30 based on search results. Moreover, it associates the classification thereof and the template (recorded in the storage unit 32) with the operation procedure (take-in/delete) according to the classification of medical information, and makes a list of the associated templates and the associated operation procedures.

### (S104)

On the monitor 14, information of the relative patient (patient ID and name) and the list generated by the list generating unit 25 are displayed. In the case of patient 1, as the information planned to be recorded in the recording unit 30, the classification of medical information (letter of introduction and images) is displayed as a list 17 (refer to FIG. 5). Moreover, on the monitor 14, by associating with the classification of medical information, a template example “T1,” “T2,” and operation procedures “take-in,” “delete” are displayed, allowing for specification. Moreover, the template example “T1” and “T2” are link sources and are associated with the address (link) of the storage area of the storage unit 32 which the template example recorded. Furthermore, details regarding what actions are performed when the template examples “T1,” “T2” and the operation procedures “take-in,” “delete” displayed on the monitor 14 are specified, are mentioned later.

### Second Embodiment

In the above, the medical system attempting solidity of the information was explained. The medical system thereof conducted actions from the extraction of information that should be extracted (S101) to the list display (S104).

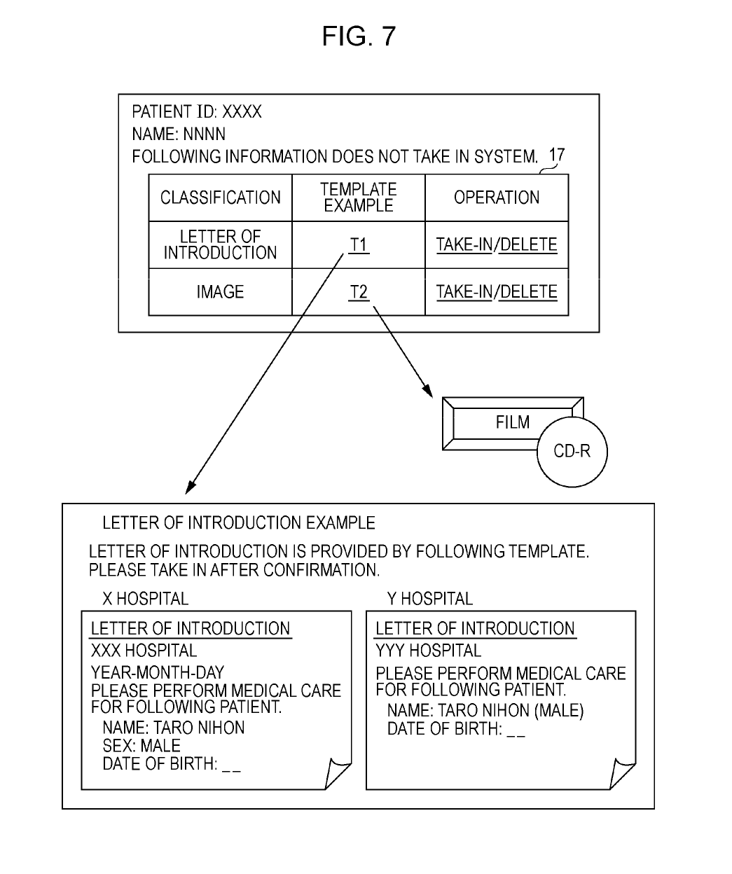

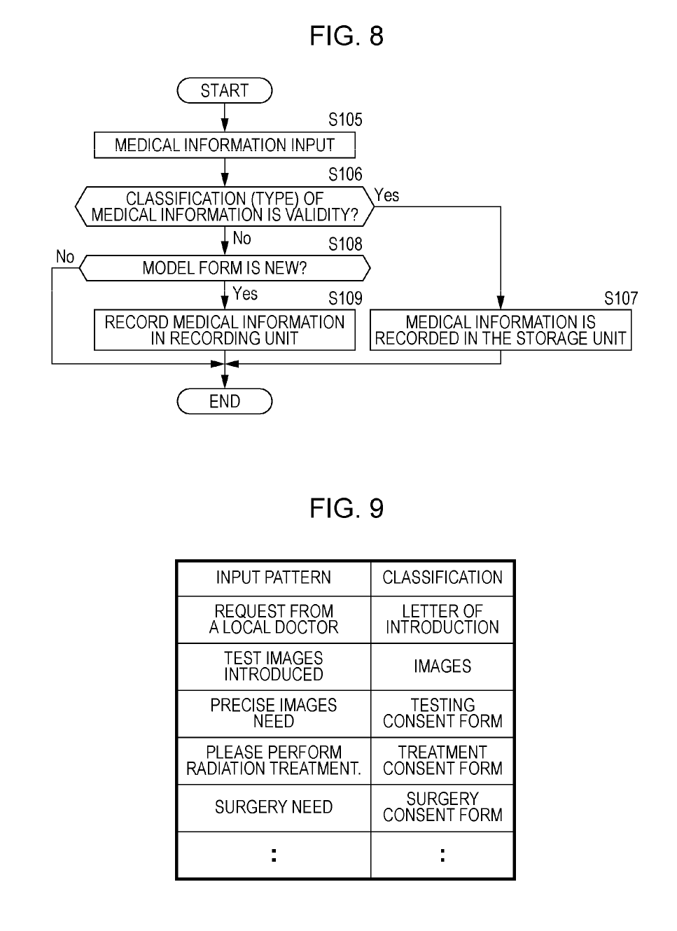

Next, the medical system attempting validity of the information is explained with reference to FIGS. 7 and 8. FIG. 7 is a diagram showing an example of the template displayed by specifying the template example in the medical system, and FIG. 8 is a flow chart showing the actions from the medical information input to the recording of the medical information.

This medical system conducts actions following the list display (S104), from the medical information input (S105 shown in FIG. 8) to recording the medical information (S109 shown in 8).

This medical system comprises a storage unit 32 that associates the classification of medical information (letter of introduction, images, etc.) and the model form thereof (template) and records this in advance, while a display unit 26 that receives specifications on the classification of medical information that should be taken into the system displayed on the monitor 14 allowing for specification, reads the model form of the medical information thereof from the storage unit 32, and displays the read model form on the monitor 14. Thereby, the user may determine whether or not the medical information taken into the system conforms to the model form thereof, and may associate this on a necessary basis upon medical practice. Here, the storage unit 32 is an example of the “management unit.” Moreover, the display unit 26 is an example of the “presenting unit.”

As information that should be taken into the system, the classification of medical information (letter of introduction, images) is displayed as a list 17 (refer to FIG. 7). The model form of the letter of introduction and the shape of the film/CD-R displayed on the monitor 14 by receiving specifications on respective template examples “T1,” “T2” from the input unit 15 are shown in FIG. 7.

The monitor 14 displays the medical information to be taken in and the model form (template) example thereof in addition to displaying the operation procedures “take-in,” “delete,” allowing for specifications (refer to FIG. 7). Specifications on “take-in” are received from the input unit 15, shifting to the intake actions in order to take in the medical information. Here, as an example of the intake actions, the location of medical information to be recorded is predetermined inside the recording unit 30, and an association is generated between the location thereof and the medical information to be taken in. On the other hand, empty links are deleted by receiving a specification of “delete” from the input unit 15.

Next, actions from medical information input to recording the medical information are explained with reference to FIG. 8.

### (Medical Information Input: S105)

When a paper media document is computerized using the scanner 16, etc. from the operation of the user (operator), or when digital data is taken in from a CD-R, etc., the transmitting and receiving unit 11 transmits the data to the server 20.

### (Differentiation of the Validity Regarding the Classification (Type) of Medical Information: S106)

The determining unit 28 determines whether or not the transmitted data (data taken in) is the medical information of the relevant patient and whether or not the data taken in is legitimate as a medical information classification that should be taken in. In the case of patient 1, for example, the letter of introduction was taken in as medical information the operator should take in; however, when explaining cases when the model form of the actual data taken in is different or when the patient name is different, the determining unit 28 matches the classification (type) of actual data taken in and the model form of the medical information recorded in advance in the storage unit 32, and determines whether or not the data belongs to patient 1. When the classification (type) of the actual data taken in differs from the model form, a message (not illustrated) is displayed on the monitor 14 as a warning.

### (S107)

When the determining unit 28 determines that the classification (type) of data taken in is legitimate (S106: Yes), the actual data is recorded in the storage unit 32 in addition to recording the information of the relevant patient and location of the actual data in the database 31. At this time, the respective information of the data taken in, information that identifies the data as being from outside, a preparation source and information source of the data thereof, are supplemented to the data taken in.

Whether or not the information pertaining to the patient is taken into the system is extracted in steps S101 to S107 and the operator is informed when the information is not taken in, thereby eliminating leakage in obtaining information of the relevant patient and allowing information necessary for medical care to be managed in the system.

In the second embodiment explained above, a determination is made regarding whether or not the information taken into the system conforms to the information that should be taken in, and may be associated on a necessary basis upon medical practice.

In the example mentioned above, a case when patient 1 received medical examination through referral from another institution was explained. As an embodiment, for example, it is hypothesized that a patient 2 underwent tests of chest CT and/or MR. Here, the “patient 2” is the identification number attached as the patient ID shown in FIG. 3. Suppose that patient 2 underwent the modality “CT, MR” and medical care of the site, “chest.” The classification of medical information associated with this situation section is “ECG data” (refer to FIG. 2).

In S101, the ECG data is extracted as information that should be taken into the system of patient 2. When the ECG data is not taken in, with steps S102 to S107, the ECG data is taken into the system as information of patient 2. In these examples, the explanation was based on the combination of the situation section shown in FIG. 2 and the classification of medical information; however, it is not limited to this example. The combination of the situation section and the classification of medical information is extended; thereby, the necessary information of the relative patient (information that should be taken into the system) may be recorded without leakage by the steps mentioned above.

### (S108)

In the description above, a case was explained in which the medical information taken in of patient 2 and the model form (template) thereof to be compared are recorded in the storage unit 32.

Next, a case is explained in which the model form (template) to be compared with the medical information of the patient that is taken in is not recorded in the storage unit 32. Suppose that the patient was referred from an institution Z. Though the model form (template) of the letter of introduction from the institution X and institution Y are recorded in the storage unit 32, the model form of the letter of introduction of the institution Z is not recorded in the storage unit 32 (refer to FIG. 4); therefore, the determining unit 28 compares the classification (type) of letter of introduction of the institution Z with the model form of the letter of introduction shown in FIG. 4, and determines that the classification (type) of letter of introduction of the institution Z is not legitimate (S106: No). Subsequently, the determining unit 28 determines whether or not the letter of introduction from the institution Z is new.

### (S109)

When the determining unit 28 determines that the classification (type) of letter of introduction from the institution Z is new (S108: Yes), it makes the letter of introduction anonymous, and with the letter of introduction from the Institution Z as the model form, or by preparing the model form of the letter of introduction of institution Z, records this in the storage unit 32; or, the model form of the letter of introduction is prepared and recorded. Moreover, locations of the supplementary information of the patient, the medical information of the patient (including the model form of the letter of introduction from the institution Z) are recorded in the database 31 and the medical information of the patient is recorded in the storage unit 32. Necessary templates may be appropriately added by conducting such processes. Meanwhile, if the letter of introduction from the institution Z is not new (S108: No), the process is completed.

### Third Embodiment

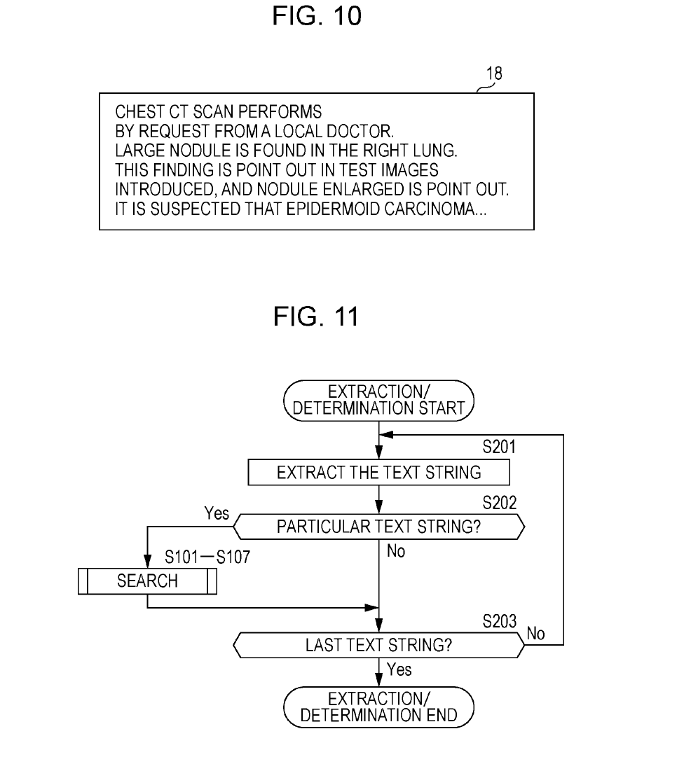

Next, a medical system attempting practical use of information is explained with reference to FIGS. 9 to 11. FIG. 9 is a diagram showing an example of the combination of input pattern and the classification of medical information recorded in the database, FIG. 10 is a diagram showing the input example of an interpretation report (survey reports), and FIG. 11 is a flow chart showing the actions of text string extraction/decision.

The medical system related to the embodiment mentioned above required inputting of the combination of the classification of medical information and the situation section recorded in the database 31 to the searching unit 24 in order to detect whether or not the classification of medical information is recorded in the recording unit 30; however, the medical system related to the third embodiment automatically searches the searching unit 24 based on inputs prepared with an application. Accordingly, even when the combination of the classification of medical information and situation section is not recorded in the database 31, whether or not the classification of medical information is recorded in the recording unit 30 may be searched, and moreover, in the application utilizing medical information, whether or not the medical information of the person associated with the input information is taken into the system may be searched easily. Here, the text string is relevant as an example of the input pattern. In such cases, in the example of the application mentioned above, software to prepare the interpretation report (survey reports) is used.

This medical system comprises an interpretation report preparing device (there are times in which the client or the server serves both) to prepare interpretation reports based on the medical information, the database 31 with association of a particular input pattern (text string) and the classification of medical information established in advance, and an extracting/determining unit 12. The interpretation report prepared by the interpretation report preparing device is recorded in the storage unit 32. Moreover, the situation section is supplemented with the interpretation report. An example of the combination of the particular input pattern (text string) and the classification of medical information recorded in the database 31 is shown in FIG. 9. For example, the combination of the input pattern “request from a local doctor” and the classification of medical information “letter of introduction” are recorded in the database 31.

The extracting/determining unit 12 reads the interpretation report from the storage unit 32, extracts the input pattern (text string), and determines whether or not the extracted input pattern is of the particular input pattern associated with the predetermined classification of medical information. The searching unit 24 searches whether or not the medical information is taken into the recording unit 30 based on the classification of medical information associated with the particular input pattern and the situation section supplemented to the interpretation report. This extracting/determining unit 12 is an example of the “extracting/determining unit.”

Next, the extracting/determining action of the text string is explained with reference to FIG. 11. In this example, a case is explained in which the combination of the classification of medical information and the situation section related to a patient 3 is not recorded in the recording unit 30. Here, “patient 3” is the identification number attached as the patient ID shown in FIG. 3.

For example, the operator (radiologist) inputs the written reports to the test images of patient 3. An example of this written report is shown in FIG. 10 as an interpretation report 18.

### (S201)

The extracting/determining unit 12 successively analyzes the written reports input by the operator and sequentially extracts the text string (input pattern).

### (S202)

Next, the extracting/determining unit 12 requests acquisition of the input pattern recorded in the database 31 to the transmitting and receiving unit 11, and determines the returned input pattern and the classification of medical information. In the case of written reports (interpretation reports) of FIG. 10, the classification of medical information corresponding with the input pattern of FIG. 9, “request from a nearby doctor,” is the “letter of introduction.”

If the text string extracted from the extracting/determining unit 12 is the same text string (particular text string) as the input pattern recorded in the database 31 (S202: Yes), the same processes as processes S101 to S107 are conducted.

### (S203)

On the other hand, when the text string extracted from the extracting/determining unit 12 is not a particular text string (S202: No), a determination is made regarding whether or not the text string is the last. Moreover, after the same processes as the processes of S101 to S107 are conducted, a determination is made regarding whether it is the last text string.

If the text string is not the last (S203: No), return to the extraction of text string (S201). Meanwhile, if the text string is the last (S203: Yes), complete extraction/decision.

According to the step mentioned above, even when there is no corresponding combination of the situation section and the medical information of the patient, the fact that there is medical information of the patient to be taken into the system from outside may be recorded in the system in advance based on the input pattern of the interpretation report (survey reports).

In the example mentioned above, the text strings input by the operator are successively analyzed and displayed as a list to be taken in. Regarding the timing thereof, for example, analysis may be conducted when the interpretation report is confirmed, recorded as information to be taken in, and the list may be displayed when the operator is viewing the information of the relevant patient, with the timing of the processes not limited.

### Fourth Embodiment

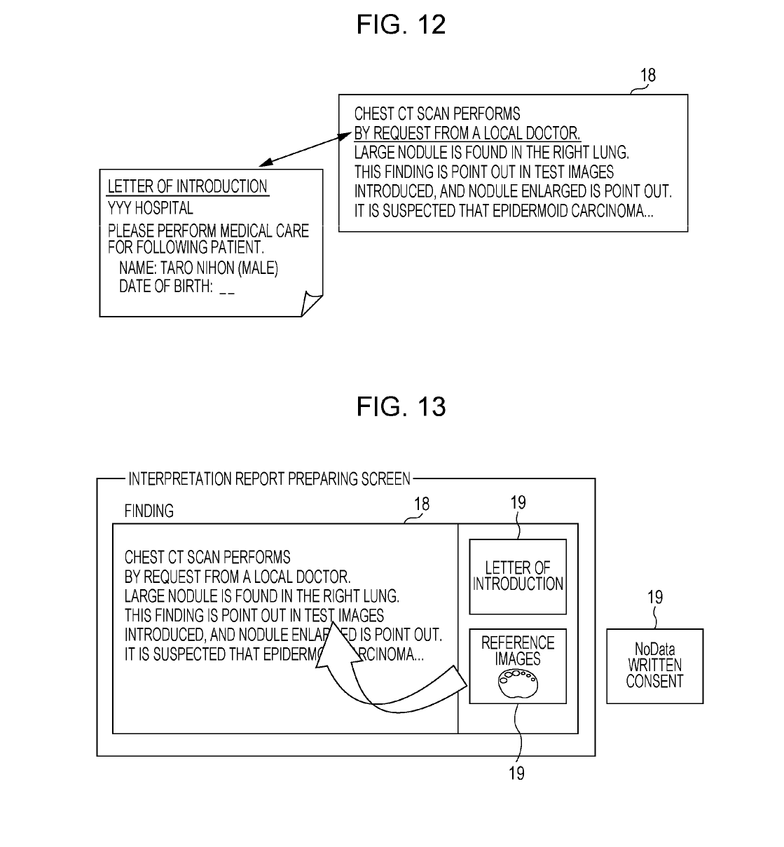

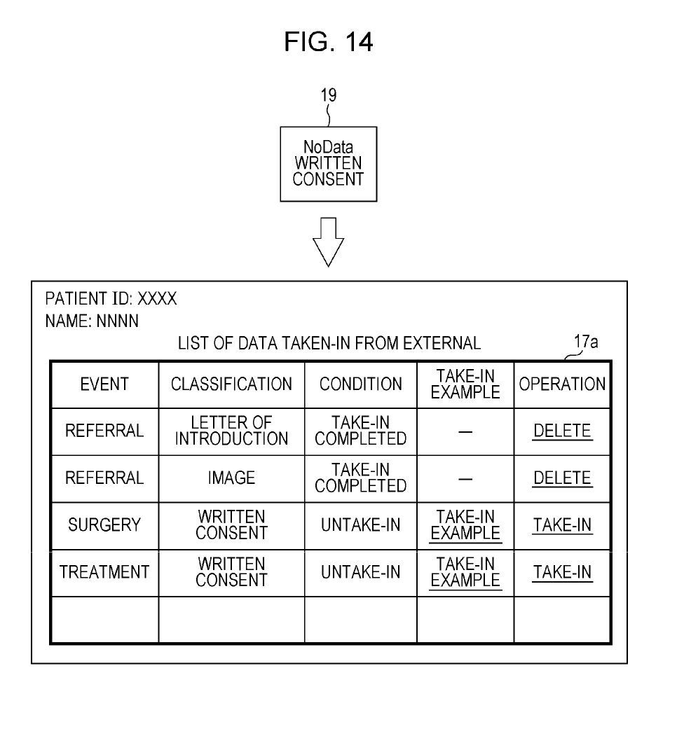

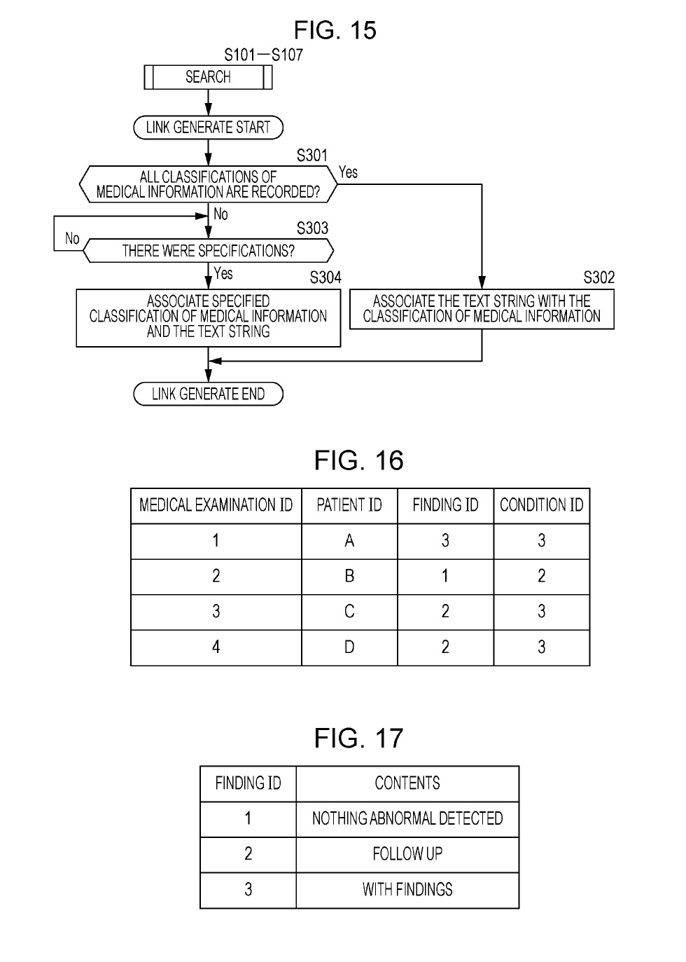

Next, other medical systems attempting practical use of the information are explained with reference to FIGS. 12 to 15. FIG. 12 is a diagram conceptually showing an example of the association of text strings and medical information, FIG. 13 a diagram showing other examples of the association of the text strings and medical information, FIG. 14 is a diagram showing an example of when the data subject for association is not taken in, and FIG. 15 is a flow chart showing the actions of link generation.

In the third embodiment mentioned above, the text string included in the interpretation report is extracted, and based on the determination that it is the classification of medical information associated with the extracted text string, whether or not the classification of medical information thereof is recorded in the recording unit 30 is searched. In contrast, this medical system was configured such that an association may be easily made between the text string and the location of medical information associated with the text string thereof.

The link generating unit 27 in order to associate the text string with the location of medical information is comprised. The link generating unit 27 has different methods of associating depending on the outcome of searching by the searching unit 24.

Next, the operations of link generation are described with reference to FIG. 15.

As a result of searching with the searching unit 24, when all classifications of medical information associated with the situation section are recorded in the recording unit 30 (S301: Yes), the link generating unit 27 associates the text string with the classification of medical information (S302).

Meanwhile, as a result of searching, when a part or all classifications of medical information associated with the situation section are recorded in the recording unit 30 (S301: No), a part or all classifications (types) of medical information are displayed on the monitor 14, allowing for specification. Next, the link generating unit 27 determines whether or not there were specifications from the input unit 15 (S303), and when it is specified within the predetermined time (S303: Yes), the specified classification of medical information and the text string are associated (S304). Meanwhile, when specification is not conducted within the predetermined time (S303: No), the link generating unit 27 determines again whether or not specification was made by the input unit 15 (S303).

Next, the action of associating with the link is explained with reference to FIGS. 12 to 14. FIG. 12 conceptually shows the association of the location of the classification of medical information (letter of introduction) associated with the text string in the interpretation report 18, “request from a nearby doctor.”

Moreover, when there is no destination of association, the link generating unit 27 generates a link without destination of association (empty link: NoData in FIG. 14), and may conduct association when the medical information is subsequently taken in. An empty link refers to predetermining the location of the medical information and associating the location thereof with the medical information that is subsequently taken in. By means of operating links generated in this manner, the list of medical information that should be taken in is displayed and presented to the user as medical information that should be taken in. The link generating unit 27 receives specifications from the input unit 15 on the presented medical information, and associates the specified medical information with the predetermined location.

The interpretation report 18 from the chest CT scan when medical care was conducted and a pattern 19 of the classification of medical information are displayed on the interpretation report preparing screen (refer to FIG. 13). The classification (type) of information displayed together with the interpretation report is searched as follows. The searching unit 24 obtains the situation section from the patient ID related to the interpretation report thereof, and searches whether or not the obtained situation section and the associated classification of medical information are recorded in the recording unit 30.

As a result of searching by the searching unit 24, the classification of medical information “letter of introduction,” “reference images” recorded in the recording unit 30 and the classification of medical information “written consent NoData” that are not recorded in the recording unit 30, are differentiated and displayed (refer to FIG. 13).

On the contrary, regarding the classification of medical information not recorded in the recording unit 30, when the subject data is not taken in, the pattern 19 of the data not taken in and a list 17a may be displayed on the interpretation report preparing screen and intake of the data may be encouraged instead of simply displaying (refer to FIG. 14).

As shown in FIG. 13, by means of determining and displaying the classification of medical information on the interpretation report preparing screen, it becomes easier to understand whether or not the classification of medical information is recorded in the recording unit 30. Furthermore, association also becomes easier. For example, from the action of drag and drop by the input unit 15, the link generating unit 27 associates the classification of medical information and the text string in the interpretation report (refer to FIG. 13).

### Fifth Embodiment

Next, other medical systems attempting practical use of the information are explained with reference to FIGS. 16 to 20.

In the following fifth embodiment, explanations are omitted regarding configurations that are the same as the third embodiment, with mainly different configurations explained.

In the third embodiment, when the classification (type) of medial information not recorded in the recording unit is searched, this is conducted based on the association of the input pattern and the classification of medical information; however, in the fifth embodiment, this is conducted based on the association of the list medical information and the classification of medical information.

FIG. 16 is a diagram showing an example of the database of the medical examination record. As shown in FIG. 16, the patient ID, finding ID, and condition ID are recorded in the database (DB) 31 in correspondence with the medical examination ID. For example, patient ID “A”, “B”, “C”, “D”, finding ID “3”, “1”, “2”, “2”, and condition ID “3”, “2”, “3”, “3” correspond with the medical examination IDs “1”, “2”, “3”, “4.”

FIG. 17 is a diagram showing an example of the master database. As shown in FIG. 17, contents of the findings are recorded in the database 31 in correspondence with the finding ID. For example, the content of findings “nothing abnormal detected,” “follow up,” and “with findings” correspond with the finding IDs “1”, “2”, and “3.”

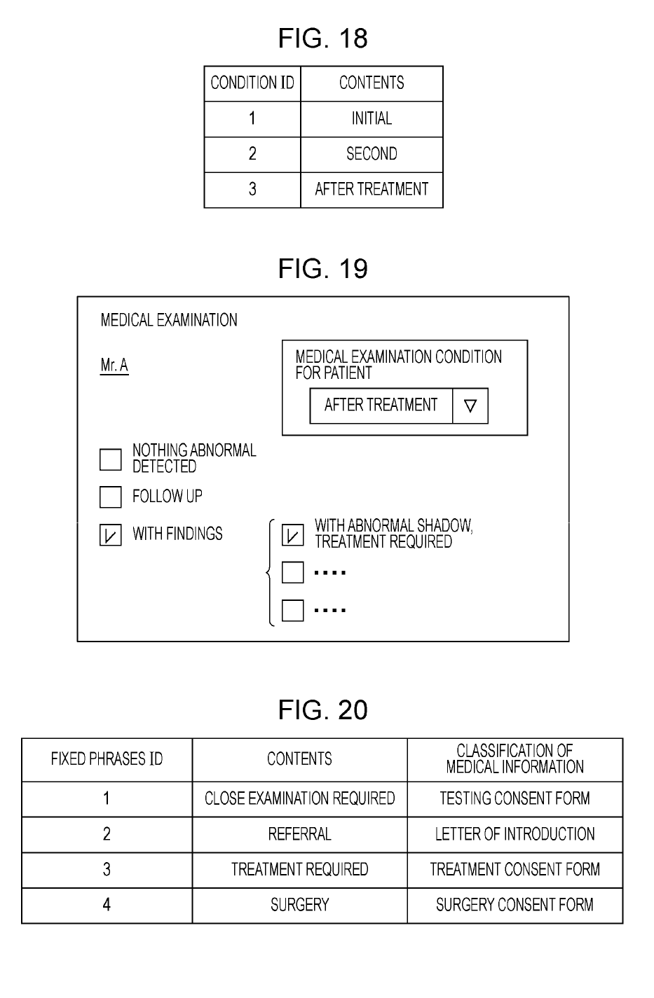

FIG. 18 is a diagram showing an example of the medical examination database. As shown in FIG. 18, the condition contents are recorded in the database B31 in correspondence with the condition ID. For example, condition contents “initial,” “second,” and “after treatment” correspond with the condition IDs “1”, “2”, and “3.” This condition content is an example of the list of medical information of this embodiment.

FIG. 19 is a diagram showing an example of an item (condition content) of medical information displayed on the monitor 14 of an interface 13.

The interface 13 comprises a display controlling unit (not illustrated) in order to display items of the medical information (condition content) on the monitor 14 allowing for selection from the specifications of the input unit 15.

On the screen of the monitor 14 shown in FIG. 19, the patient ID “A” corresponding with the medical examination ID “1,” the condition content “after treatment” corresponding with the condition ID “3,” and the content of findings “with findings” corresponding with the finding ID “3,” are displayed.

The condition content “after treatment” is displayed in the column for “medical examination condition of the patient” on the screen shown in FIG. 19. Furthermore, on this screen, the content of findings “with findings” is displayed on this screen due to a mark “✓” being added to the column of checkboxes. Furthermore, text information “with abnormal shadow, treatment required” is displayed by supplementing the contents of finding “with findings.”

FIG. 20 is a diagram showing an example of the database of fixed phrases. As shown in FIG. 20, the content of the fixed phrases of and the classification of medical information are recorded in the database 31 in correspondence with the fixed phrase ID. For example, contents of the fixed phrases “close examination required,” “referral,” “treatment required,” and “surgery” as well as the classifications (types) of medical information “test consent form,” “letter of introduction,” “treatment consent form,” and “surgery consent form” correspond to the fixed phrase IDs “1”, “2”, “3”, and “4.”

In other words, the text information “with abnormal shadows, treatment required” displayed as a supplement to the content of findings “with findings” shown in FIG. 19 corresponds with the content of the fixed phrase “treatment required” shown in FIG. 20. Furthermore, association is made with the classification of medical information “treatment consent form” from the fixed phrase ID “4.”

In the manner described above, when the condition content “after treatment” is selected by the input unit 15 as the item of medical information related to patient ID “A” on the monitor 14 of the interface 13, the management unit (database 31 and the storage unit 32) associates and manages the item of medical information (condition content) “after treatment” and the classification of medical information “treatment consent form.”

Next, the method of searching the classification of medical information that is not recorded in the recording unit is explained.

First, the extracting/determining unit 12 reads the interpretation report from the storage unit 32, and determines whether or not the item of medical information related to the predetermined patient, for example, patient ID “A,” for example, the condition content “after treatment,” is a medical item associated with the classification of medical information. Thereby, the extracting/determining unit 12 extracts the classification of medical information “treatment consent form.”

Next, under the condition of medical information of the extracted classification, “treatment consent form,” not recorded in the recording unit (database 31 and/or storage unit 32), the searching unit 24 searches the classification of medical information that satisfies the condition thereof.

Next, the display control unit of the interface 13 displays the classification of medical information searched by the searching unit 24 on the monitor 14.

In the fifth embodiment mentioned above, the extracting/determining unit 12 determines the necessary medical information in correspondence with the medical examination situations of the patient, the searching unit 24 searches the system, and when the medical information is not taken into the system, it may inform this to the operator as medical information that should be taken in.

Furthermore, in the embodiment mentioned above, the extracting unit 23 or the extracting/determining unit 12 were shown as the unit to determine whether or not the medical information is necessary in accordance with the medical examination situations of the patient; however, these unit may be provided on any of the client 10 or the server 20.

According to the various embodiments mentioned above, it is encouraged that the information be the necessary medical information corresponding to medical examination situations, and may be managed. Moreover, referring to the medical information of the patients obtained from outside the system becomes easy, and improved operation effectiveness may be hoped for.

Several embodiments of the present invention have been described, but these embodiments have been presented as examples and are not intended to limit the scope of the invention. These new embodiments may be implemented in various other modes, and various omissions, substitutions, and changes may be made within the scope of the substance of the invention. These embodiments and modifications thereof are included in the scope and substance of the invention and are also included in a scope equivalent to that described in the scope of patent claims.

### EXPLANATION OF SYMBOLS

### 10 Client

11 Transmitting and receiving unit
12 Extracting/determining unit

### 13 Interface

### 14 Monitor

15 Input unit

### 16 Scanner

### 20 Server

21 Transmitting and receiving unit
22 Process controlling unit
23 Extracting unit
24 Searching unit
25 List generating unit
26 Display unit
27 Link generating unit
28 Differentiating unit
30 Recording unit

### 31 Database (DB)

32 Storage unit

### 40 Modality

1-7. (canceled)

8. A medical system, comprising:
a memory that records medical information by associating the medical information with classification thereof:
a management unit that associates and manages a text string input pattern and the classification of medical information;
an extracting unit that extracts medical information that should be recorded as information of a predetermined patient based on the text string input pattern and the classification of medical information associated by the management unit, and text strings comprised in the medical information of the predetermined patient;
a searching unit that searches the classification of medical information related to the predetermined patient not recorded in the memory among the classification of medical information extracted by the extracting unit; and
a display that displays the classification of medical information searched by the searching unit.

9. The medical system according to claim 8, further comprising a link generating unit that associates the text string comprised in the medical information of the text string and a predetermined location in which the classification of medical information associated with the input pattern related to the text string is recorded by the memory.

10. The medical system according to claim 9, wherein, as a result of the searching, when apart or all classifications of medical information associated with a situation section by the management unit is not recorded in the memory, the part or all classifications of medical information is displayed on the display unit allowing for specification, and is specified by the input unit, the link generating, unit associates the specified classification of medical information with the text string.

11. The medical system according to claim 8, wherein the memory records the medical information prepared outside of the system by associating the information of an information source to determine whether or not the medical information is from outside, information source identification information to differentiate the information source, and preparation source identification information to identify with the preparation source.

12. The medical system according to claim 9, wherein the memory records the medical information prepared outside of the system by associating the information of an information source to determine whether or not the medical information is from outside, information source identification information to differentiate the information source, and preparation source identification information to identify with the preparation source.

13. The medical system according to claim 10, wherein the memory records the medical information prepared outside of the system by associating the, information of an information source to determine whether or not the medical information is from outside, information source identification information to differentiate the information source, and preparation source identification information to identify with the preparation source.
